(19) United States
(12) Patent Application Publication
(10) Pub. No.: US 2008/0071247 A1
(43) Pub. Date: Mar. 20, 2008

(54) Topical administration of a salt compound to facilitate venipuncture

(75) Inventor: R. Kent Hermsmeyer, Portland, OR (US)

Correspondence Address: HOWARD EISENBERG, ESQ., 1220 LIMBERLOST LANE, GLADWYNE, PA, 19035, US

(73) Assignee: Dimera Incorporated

(21) Appl. No.: 11/880,137

(22) Filed: Jul. 21, 2007

Related U.S. Application Data
(60) Provisional application No. 60/845,473, filed on Sep. 18, 2006.

(51) Int. Cl. A61M 35/00

(52) U.S. Cl. 604/507; 604/290

(57) ABSTRACT

A method for enlarging a peripheral vein so as to facilitate venipuncture of the vein by topically applying a vasodilator salt compound to skin overlying the vein and permitting the salt compound to cause the vein to enlarge by causing the vein to fill with blood.

### RELATED APPLICATIONS

This application claims the benefit of the priority date of U.S. Provisional Patent Application Ser. No. 60.845,473, filed on Sep. 18, 2006.

### FIELD OF THE INVENTION

The invention pertains to the field of cutaneous venipuncture, particularly to the field of rendering cutaneous veins more accessible for venipuncture.

### BACKGROUND OF THE INVENTION

Venipuncture is the act of puncturing a vein for surgical or therapeutic purposes or for the collection of blood specimens, such as for analysis. It is a routine technique that is performed countless times every day in hospitals and clinics throughout the world.

When performing a venipuncture in the antecubital region, a phlebotomist typically swabs the phlebotomy site with a cleansing agent, such as alcohol, and places a tourniquet on a subject's arm above the elbow. The tourniquet is tightened sufficiently to retard venous blood flow in the arm which causes blood to pool in the veins of the arm. This results in an increase in the firmness and in diameter of the vein of interest which facilitates venipuncture at that site. A similar technique is typically performed for venipuncture of a vein on the dorsal surface of the hand.

This standard technique has several distinct disadvantages. Alcohol, either ethyl or isopropyl, although useful in aiding visualization of a cutaneous vein and as an aid in cleansing the skin overlying a vein, is a vasoconstrictive agent that actually induces a diminution of the size of the vein. In addition, the use of tourniquets is contraindicated in many situations and the prolonged or repeated use of a tourniquet may result in unwanted complications.

With many individuals, especially children, the elderly, and people with small veins or low blood pressure, venipuncture is difficult because the target vein fails to fill adequately. Several techniques may be attempted if a suitable vein cannot readily be obtained. These include tightening the tourniquet, massaging the arm between the wrist and the elbow to augment venous return from the more distal portions of the limb, lowering the arm to induce pooling of blood in the limb, and applying heat to the venipuncture site. Although these methods may occasionally be successful, many times a suitable vein cannot be found even following the application of these techniques. Moreover, these types of additional techniques such as tightening a tourniquet or applying heat to a patient's limb may have unintended adverse consequences.

In laboratory animals, such as rats, mice, and rabbits, dilation of a vein for venipuncture has been obtained by swabbing the skin overlying the vein site with xylene. The use of xylene in laboratory animals is discouraged, however, by many institutions because it causes severe irritation. Because of its property as an irritant, xylene is unsuitable for use for cutaneous application as an aid in venipuncture in humans and in veterinary patients. Accordingly, xylene is expressly excluded from the scope of the present invention.

Birnbaum, U.S. Pat. No. 4,311,707, discloses that natural and synthetic prostaglandins, when administered topically to the skin, produce a distinct increase in cutaneous and peripheral circulation. The topical administration of the prostaglandins may produce a distinct and persistent erythema at the site of application. According to Birnbaum, the topical administration of prostaglandins is useful for local treatment of peripheral vascular diseases of the arteriospastic and occlusive types, such as Buerger's disease, livedo reticularis, acrocyanosis, atherosclerosis, frostbite, and impending gangrene. Birnbaum further discloses that topically administered prostaglandins may be useful to enhance healing of wounds, ulcers, infections, proliferative and inflammatory skin lesions, to treat impotency, or to enhance the rate of absorption of pharmaceutically active agents. Birnbaum does not disclose the topical administration of a prostaglandin to a venipuncture site or for facilitation of venipuncture, nor does Birnbaum disclose an increase in size or firmness of veins in the region of administration of the prostaglandin.

Several studies have shown that topically applied nitroglycerin produces enlargement of dorsal hand veins and facilitates venipuncture. Other studies have shown similar results with isosorbide dinitrate. The use of these drugs in this way has the disadvantage that topical application of these drugs produces systemic effects, including hypotension and headaches. Therefore, the topical application of these drugs is not practical for routine venipuncture.

Frequently, prior to venipuncture, the skin over superficial veins is often cleansed with a swab impregnated with ethyl or isopropyl alcohol. These agents are useful as disinfectants and as cleansing agents. They also aid in visualization of the vein underlying the skin. However, both ethyl and isopropyl alcohol are vasoconstrictors and so do not cause enlargement of superficial veins.

A significant need exists for a method by which veins, especially those in individuals having difficult-to-access veins, can be enlarged and, therefore, made more readily accessible for venipuncture.

### DESCRIPTION OF THE INVENTION

It has been discovered that cutaneous topical application of a salt compound to a venipuncture site facilitates venipuncture of a vein at that site. The application of the salt compound induces pooling of blood within the vein located at the venipuncture site with an increase in diameter and turgor of the vein. In this way, a desired vein is both easier to find and easier to puncture with a needle.

In one embodiment, the invention is a method for increasing the accessibility of a vein for venipuncture. According to this embodiment, a salt compound is topically applied to the skin overlying a vein, thereby causing the vein to enlarge and become more readily accessible for venipuncture. The salt compound may be administered in combination with another chemical compound that serves to cleanse and/or disinfect the site.

The term “salt compound” as used herein means a chemical compound in which all or some of the hydrogen ions of a corresponding acid have been replaced with metal ions or electropositive radicals. As used herein, the term “salt compound” includes chemical compounds in a solid state and in a dissolved state. When dissolved in an aqueous fluid, the dissolved ions of the salt compound conduct electricity.

The term “enlarge” or “enlargement” as used herein means a visibly observable increase in size and pressure of one or more distinct superficial veins with an increase in elevation of the top surface of the veins as the pressure increases. Enlargement is in contrast to a generalized erythema and indicates an increase in diameter and turgor of the vein. This is analogous to the increase in vertical size of a soft-sided garden or fire hose when a water tap, to which the hose is attached, is opened.

As used herein, the terms “vasodilator” refers to a compound that causes enlargement of superficial veins in accordance with the method of the invention.

In another embodiment, the invention is a method for performing venipuncture in a subject. According to this embodiment, a site for venipuncture is selected, a salt compound is topically applied to the skin overlying a vein at that site, the vein is permitted to enlarge as a consequence of the application of the salt compound, and the vein is then punctured.

In another embodiment, the invention is an article of manufacture, such as a kit, for facilitating venipuncture in a subject. According to this embodiment, the article of manufacture or kit contains a package housing a container containing a topical preparation containing one or more salt compounds for topical application, an applicator for application, such as by rubbing or swabbing, of the salt compound on the surface of the skin, and optionally instructions for application of the salt compound to the skin to prepare a vein for venipuncture. In a preferred embodiment, the applicator is a fabric swab that is impregnated with the salt compound and the container is a sealed foil-lined disposable pouch that is torn open by the user to break the seal and provide access to the swab.

The invention in all its embodiments is useful for facilitating venipuncture in a subject, such as a mammalian or avian subject. The subject may be a human subject or a veterinary subject, such as a domestic pet animal like a dog, cat, or bird, a farm animal such as a horse, cow, goat, sheep, llama, or pig, or a non-domesticated mammal or bird.

The site of venipuncture suitable for the invention is a site on the skin of a subject at which site a vein underlying the skin is in a position suitable for venipuncture. Preferably, the vein is visible through the skin before application of the vasodilator salt compound in accordance with the invention. Alternatively, but not preferably, the vein is not visible through the skin of the subject but, upon application of the salt compound, the vein becomes visible.

Any vein that is suitable for venipuncture in a human or other animal is suitable for the method of the invention. Preferred veins include but are not limited to those of the antecubital region, such as the basilic, cephalic, and median cubital veins, and those found on the back of the hands or wrist. Other suitable but less preferred veins include but are not limited to those situated in the legs such as at the top of the feet, the ankle, or the femoral region, and in the neck, such as the jugular vein. Additional veins that are more suitable in animals other than humans include the veins on the udder, the vein on the ventral aspect of the tail, primarily used in cattle, and ear veins primarily used in rabbits and pigs.

The salt compound may be applied to the skin of a subject at a desired venipuncture site by any means by which a chemical compound may be topically applied. Preferably, the salt compound is in liquid form, preferably in aqueous or non-aqueous solution, such as an alcohol-based solution. The salt compound may be combined with other chemical compounds such as disinfectants like alcohol or povidone-iodine, buffers, or solubilizing agents. Examples of suitable means of application of the salt compound include by spraying, painting, dabbing, wiping, and dripping, immersing, massaging, swabbing, and rubbing.

The amount of the salt compound that is applied in accordance with the invention is an amount that is effective to produce an enlargement of a vein situated under the skin of a subject at the site of application of the salt compound. The amount that is effective to produce such a desired effect depends on several factors, including the selected site for venipuncture, the thickness of the skin at the selected venipuncture site, the medical status of the subject, the concentration of the vasodilator salt compound that is applied to the site, and the identity of the salt compound that is applied. Generally, it is conceived that the salt compound is in a concentration such that a volume of 3 ml or less will be effective when applied to the site. Preferably, the volume applied is less than 3 ml, more preferably less than 2 ml, and most preferably about 1 ml or less.

In a preferred embodiment, a liquid containing a salt compound is impregnated within an absorbent fabric such as in the form of a swab, a gauze pad, or a cotton ball. Depending upon the nature of the salt compound and the presence and nature of a solvent for the salt, the fabric may be a woven or non-woven fabric and may be water absorbent, such as a cellulosic fabric like cotton, or may be non-water absorbent, such as an olefin. The salt compound impregnated in the fabric is applied by contacting the fabric to a desired venipuncture site, such as by dabbing, wiping, painting, or rubbing the absorbent fabric onto the skin of a subject at the site. The salt compound is permitted to provide its effect of enlarging a vein underlying the skin at the site, facilitating venipuncture of the vein.

The salt compound that is suitable for the invention is one that is a salt of a cation of an element that has an atomic number less than 57 and that, when ionized, has a valence of +1, +2, or +3 and that, when applied topically to the skin of a subject at a site overlying a vein, (1) is a vasodilator, which means that it will cause enlargement of the vein within 5 minutes of application when applied alone or when applied in combination with one or more chemical compounds will cause increased enlargement of the vein compared to the enlargement produced when the one or more chemical compounds are applied without the salt compound, (2) is locally non-irritating, and preferably (3) does not produce systemic biochemical effects following topical application to the skin of a subject. That is, application of the salt compound to the skin of a subject in an amount that is effective in accordance with the method of the invention does not produce an inflammatory reaction in the skin of the subject and preferably does not produce observable systemic effects in the subject.

Vasodilator salt compounds that are suitable for the invention include inorganic and organic salts. In a preferred embodiment, the salt compound is an inorganic salt, which is preferably a divalent cationic salt of an element, such as a salt of magnesium, calcium, or copper. Monovalent or trivalent cationic salts are also suitable for the invention. Specific examples of suitable inorganic salts include sulfates, sulfites, nitrates, phosphates, and halides such as chlorides. In another preferred embodiment, the salt is an organic salt of a monovalent, divalent, or trivalent inorganic cation. Preferred organic salts include acetate and citrate salts.

Combinations of two or more vasodilator salt compounds as described above or of one or more such salts and one or more additional chemical compounds may increase the enlargement of veins that is produced compared to that produced when only one such salt is applied. For example, applying benzalkonium chloride, povidone-iodine, or a cyclodextrin such as hydroxypropyl-betacyclodextrin, in combination with a salt compound may increase the vasodilation produced by that salt compound when applied alone.

Moreover, in some instances, the combination of a salt compound and one or more chemical compounds, such as the combination of a salt compound and cyclodextrin, benzalkonium chloride, or povidone-iodine may be a vasodilator that is suitable for the invention whereas the salt compound without the cyclodextrin, benzalkonium chloride, or povidone-iodine, would not be a vasodilator that is suitable for the invention. In such instances, the combination of the salt compound with the additional chemical compound produces an enlargement of veins that is increased compared to that obtained when the chemical compound is used without the salt compound.

In accordance with the article of manufacture or kit of the invention, one or more salt compounds as described above is contained within a container, such as a bottle or a tear-open foil-lined disposable pouch, which container may be housed within a package. The package further contains an applicator, such as a dropper, a cotton ball, a spray mechanism, or a swab, for applying the salt compound to the skin of a subject. The applicator may be situated within the container that contains the salt compound, for example if the container is a foil-lined pouch and the chemical compound is impregnated in a fabric swab that functions as an applicator. Alternatively, the applicator may be situated outside of the container containing the salt compound, for example if the applicator is a cotton ball that is impregnated with the vasodilator chemical compound just prior to application of the salt compound to the skin of a subject. Preferably, the kit further contains written instructions to apply the salt compound to the skin of a subject in accordance with the invention described herein. Such written instructions may be housed within the package or may be instructions on the surface of the package itself.

The invention is further described in the following non-limiting examples.

### EXAMPLE 1

Several studies were conducted to determine the effect of topically applied compounds on peripheral venous filling. The same two healthy adult male volunteer subjects were used for each of the studies.

In each study, a baseline value for peripheral venous filling time was determined as follows. Each subject held his outstretched arm vertically with hand directed upwards toward the ceiling for a period of time sufficient to cause the veins visible on the dorsum of the hand to attain their minimum dilation. Then, the subject lowered his hand and held his outstretched arm vertically with hand directed downwards toward the floor for a period of time sufficient to cause the veins visible on the dorsum of the hand to attain their maximum dilation. The time for the veins to attain maximum dilation following the lowering of the hands was measured.

This procedure was repeated following application of a test compound to the site of visible veins on the dorsum of the hands in order to determine the effect of the test compound on venous filling. Longer venous filling times are indicative of vasodilation and increased pooling of blood within the vein. The data is shown in Table 1. Results in Table 1 represent the average venous filling time of the two subjects, with no chemical compound topically applied and then with the application of a test chemical compound.

TABLE 1

| | | VENOUS | |
|---|---|---|---|
| | COMPOUND APPLIED | FILL | % CHANGE |
| | TOPICALLY | TIME (sec) | FROM BASELINE |
| STUDY 1 | None | 54 | — |
| | EtOH | 59 | 9 |
| | MgCl₂ | 158 | 190 |
| STUDY 2 | None | 60 | — |
| | Nitroglycerine (prior art) | 100 | 67 |
| STUDY 3 | None | 51 | — |
| | EtOH | 38 | (−25) |
| | MgSO₄ in EtOH | 91 | 78 |
| STUDY 4 | None | 39 | — |
| | Isopropyl alcohol | 69 | 76 |
| | MgSO₄ isopropyl alcohol | 109 | 179 |

MgCl₂ - 100 mM,
Nitroglycerine - 100 μM,
EtOH (ethyl alcohol) - 70%,
MgSO₄ - 10 mM

As shown in Table 1, baseline venous fill time varied between 39 and 60 seconds. Topical application of ethyl alcohol had no effect on venous fill time, showing an increase in fill time of 9% in Study 1 and a decrease in fill time of 25% in study 3. In contrast to ethyl alcohol, topical application of isopropyl alcohol resulted in an increased venous fill time. Combining MgSO₄ with isopropyl alcohol resulted in greater than a doubling of the change in venous fill time obtained with isopropyl alcohol alone; Application of all other compounds tested, including nitroglycerine, MgCl₂, and MgSO₄, produced substantial increases in venous filling time compared to baseline control.

### EXAMPLE 2

### Dorsal Hand Veins

Infrared (IR) thermal imaging utilizing a standardized, calibrated infrared (950 nm wavelength) camera acquiring electronic images and providing color coded maps for testing effects of test vasodilator chemical compounds on dorsal hand veins was performed in three male and two female healthy volunteers. Infrared was utilized to determine the degree to which the test chemical compounds cause vasodilation because infrared provides a measure of thermal gradient and diameter that correlates well with visual indications of increased venous size and with successful first attempt venipuncture.

Test agents were applied by swabbing the target area for about 30 seconds. A baseline control without application of any swab or by swabbing with a placebo swab containing no liquid was performed at the start of each test as a basis to compare imaging of the vein dilator dependent filling. IR images were obtained using a standardized, calibrated infrared (950 nm wavelength) camera that acquired an electronic image and provided a color coded thermal map.

### EXAMPLE 2a

### Isopropyl Alcohol

Each subject was tested for vasodilation of the dorsal hand veins with (a) a dry swab (control), (b) a commercially available swab containing 70% isopropyl alcohol (Becton Dickinson, Franklin Lakes, N.J., US), and © MgSO₄ in 70% isopropyl alcohol. The swab was rubbed onto the dorsal surface of a hand of each subject and then the hand was elevated for 2 minutes after which the hand was returned to a horizontal position. IR imaging was performed on the dorsum of the hand before elevation and at intervals following the return to the horizontal position.

Compared to control, both of the test swabs (b) and © caused an initial vasoconstriction with reduced filling of the veins on the dorsum of the hand. This was shown on IR examination as a narrower thermal gradient and reduced diameter at 30 seconds following return to horizontal. The test swabs containing © MgSO₄ caused increased venodilation compared to control and compared to isopropyl alcohol alone after 5 to 10 minutes following return to the horizontal position.

These results indicate that isopropyl alcohol, when applied topically, acts as a vasoconstrictor to superficial veins. Topical application of the vasodilator MgSO₄, when formulated in isopropyl alcohol, resulted in an initial vasoconstriction followed by a venodilation. The vasoconstriction is conceived to be due to the presence of the isopropyl alcohol and the venodilation occurred only after enough time had elapsed for sufficient evaporation of the alcohol to occur which then permitted the effect of the vasodilator compound to dominate.

Because both ethyl and isopropyl alcohol act as vasoconstrictors of superficial veins, the application of either or both of these alcohols to a venipuncture site without the concurrent application of a salt compound as described above is expressly excluded from the scope of the invention. Expressly included within the scope of the invention, however, is the concurrent application of an alcohol, such as ethyl or isopropyl alcohol, and a salt compound. Such concurrent application may be simultaneous application, such as by application by using a swab that is impregnated with both an alcohol and a salt compound, or may be in succession, such as by applying a salt compound before or after, such as within one to 5 minutes, applying an alcohol to a venipuncture site.

### EXAMPLE 2b

### Absence of Isopropyl Alcohol

The protocol was repeated utilizing swabs of impregnated with either MgSO₄ (10 mM) in water or MgSO₄ (10 mM) in combination with benzalkonium chloride (1%). Results are shown in Table 2.

TABLE 2

| | Fold Increase in | Increase in Venous |
|---|---|---|
| | Diameter Compared to | Diameter Following |
| | Control | Hand Elevation |
| CONTROL (dry swab) | — | <10% |
| MgSO4 | 3 | 30% |
| MgSO4 + benzalkonium | 4 | 40% |
| chloride | | |

Venodilation obtained with topical application of MgSO₄ was rapid, within 60 to 90 seconds, and marked. Measurement of maximal dilation of superficial dorsal hand veins following application of MgSO₄ alone revealed about a 30% increase in venous diameter following return to horizontal after hand elevation. This was about greater than three times that obtained in the control subjects in which an increase in diameter of less than 10% was obtained. When benzalkonium chloride was combined with MgSO₄, the results were even more striking, producing about a 40% increase in venous diameter, or about 4 times that of the control.

### EXAMPLE 3

### Vein of Inner Elbow

Studies in Example 2 were performed except that the vein of the inner elbow, the site most frequently used for phlebotomy, was evaluated and vein diameter was measured from enlarged, sequential, visible light images. The protocol differed from that of Example 2 as the data was obtained without a hydrostatic maneuver (arm elevation and return to horizontal) because refilling in inner elbow veins, in contrast to that seen in dorsal hand veins, is not detectable. Instead the response to swabbing was recorded with the arm in a stationary position resting on a table top.

The study was performed with swabs impregnated with (a) 70% isopropyl alcohol, (b) 10 mM MgSO₄, © 1% benzalkonium chloride, (d) combination of 10 mM MgSO₄ and 1% benzalkonium chloride, (e) 10% povidone-iodine (BETADINE®, Purdue Frederick Co., Stamford, Conn.), or (f) combination of 10 mM MgSO₄ and 10% povidone-iodine. The initial image was taken prior to application of each test agent. The endpoint image was taken nine minutes following application of the test agent. The vein diameters were measured in pixels. Data is shown in Table 3.

TABLE 3

| | Diameter Pre- | Diameter Post- | |
|---|---|---|---|
| | Application | Application | |
| Test Agent | (pixels) | (pixels) | Percent change |
| 1% benzalkonium | 34.41 | 37.80 | 9.85 |
| chloride | | | |
| 10 mM MgSO₄ | 65.86 | 73.06 | 10.93 |
| 10 mM MgSO₄ + | 87.80 | 112.20 | 27.79 |
| benzalkonium | | | |
| chloride | | | |
| 10% povidone-iodine | 79.61 | 75.82 | (−4.76) |
| 70% isopropyl | 57.78 | 55.04 | (−4.74) |
| alcohol | | | |
| 10 mM MgSO₄ + | 11.18 | 12.17 | 8.81 |
| 10% povidone-iodine | | | |

The elbow veins following application with either isopropyl alcohol or povidone-iodine alone showed a decrease in size that persisted for several minutes following application. In contrast, application of MgSO₄ produced a rapid venodilation that was measurable at first measurement 30 seconds following application and increased over the next 5 minutes. Application of benzalkonium chloride produced a venodilation less than that obtained with MgSO₄ alone. Application of the combination of MgSO4 and either benzalkonium chloride or povidone-iodine produced an enhanced venodilation. Maximal dilation of inner elbow veins observed was obtained with the combination of MgSO₄ and benzalkonium chloride.

### EXAMPLE 4

### Testing of Additional Vasodilators

The procedure of Example 3 was repeated utilizing swabs impregnated with various salt compounds on one to three adult male and female subjects. Each test compound was applied to the skin overlying the vein of the inner elbow. The initial image was taken prior to application of each test agent. The endpoint image was taken nine minutes following application of the test agent. The vein diameters were measured in pixels. Data is shown in Table 4.

TABLE 4

| | AVERAGE % CHANGE IN VEIN |
|---|---|
| TEST COMPOUND | DIAMETER |
| 70% Isopropyl alcohol | −13.2 |
| 10 mM MgSO₄ | 30.0 |
| 100 mM MgSO₄ | 25.2 |
| 1 M MgSO₄ | 22.9 |
| 10 mM Mg citrate | 4.2 |
| 10 mM Mg acetate | 27.9 |
| 10 mM MgCl₂ | 6.5 |
| 10 mM CaCl₂ | 12.0 |
| 10 mM CaSO₄ | 17.7 |
| 10 mM Ca acetate | −0.5 |
| 10 mM CuCl₂ | 10.5 |
| 10 mM CuSO₄ | 20.95 |
| 10 mM SrCl₂ | −6.0 |
| 10 mM SrSO₄ | 16.95 |
| 10 mM Sr acetate | 7.35 |
| 10 mM MnCl₂ | 2.7 |
| 10 mM MnSO₄ | 0.0 |
| 10 mM Mn acetate | 0.0 |
| 10 mM ZnSO₄ | −4.4 |

As shown in Table 4, topical administration of isopropyl alcohol caused a constriction of the underlying vein. The data for magnesium sulfate shows that this compound is a powerful vasodilator and that the optimal concentration appears to be 10 mM. For this reason, a concentration of 10 mM was used for all subsequent testing.

All salts of magnesium tested were shown to be vasodilators when topically applied to the skin overlying the vein of the elbow. Of the magnesium salts tested other than the sulfate, the acetate provided the greatest degree of vasodilation, with the vasodilation provided by the chloride and citrate being less marked.

Salts of calcium, copper, zinc, strontium, and manganese were also tested. All salts of copper tested provided vasodilation. The chloride of manganese provided mild vasodilation whereas other manganese salts tested, and the zinc salt tested, failed to demonstrate vasodilation in this test. It is conceived that salts of these elements will prove to be useful as vasodilators in accordance with the present invention if tested in a larger group of subjects or with varying concentrations or anions. Similarly, although salts of calcium and of strontium provided significant vasodilation, the acetate salt of calcium and the chloride salt of strontium did not show vasodilation. It is conceived that these salts of these elements will prove to be useful as vasodilators in accordance with the present invention if tested in a larger group of subjects or with varying concentrations or anions.

The present invention has been described in terms of particular embodiments proposed to comprise preferred modes for the practice of the invention. It will be appreciated by those skilled in the art that, in light of the above disclosure, numerous modifications and changes may be made in the particular embodiments described and exemplified without departing from the scope of the invention. For example, it is well within the routine skill of one in the art to test various salts of +1, +2, or +3 cations, such as salts of sodium, potassium, silver, or aluminum, to determine whether such salts will be useful as a vasodilator in accordance with the present invention. Likewise, it is well within the routine skill of one in the art to test salts of various anions, such as nitrates, sulfites, phosphates, and halides in addition to chlorides, to determine whether such salts are suitable for the present invention. Such salts, even though not specifically disclosed herein, are intended to be included within the scope of the present invention, as defined in the claims that follow. Further, other modifications, uses, and applications of the invention described herein will be apparent to those skilled in the art. It is intended that such modifications be encompassed in the claims that follow.

1. A method for increasing the accessibility of a vein for venipuncture comprising topically applying to the surface of skin of a subject overlying a vein a composition containing an effective amount of a salt of a cation of an element that has an atomic number less than 57 and that, when ionized, has a valence of +1, +2, or +3, thereby causing the vein to enlarge and become more readily accessible for venipuncture.

2. The method of claim 1 wherein the salt is applied in combination with ethyl or isopropyl alcohol.

3. The method of claim 1 wherein 3 ml or less of the composition is applied to the skin.

4. The method of claim 1 wherein the composition is in the form of a liquid which is impregnated in an absorbent fabric and application of the composition is by contacting the fabric to the skin.

5. The method of claim 1 wherein the cation is magnesium.

6. The method of claim 1 wherein the cation is selected from the group consisting of magnesium, calcium, copper, strontium, and manganese.

7. The method of claim 1 wherein the anion of the salt is selected from the group consisting of sulfate, chloride, citrate, and acetate.

8. The method of claim 1 wherein the composition is applied to the skin by wiping, dabbing, swabbing, or rubbing a fabric swab impregnated with the composition onto the skin.

9. The method of claim 1 wherein the salt compound is applied to the skin in combination with benzalkonium chloride or povidone-iodine.

10. The method of claim 1 which further comprises permitting the vein to enlarge due to the application of the composition and then puncturing the vein.

11. A method for performing venipuncture in a human or animal subject comprising selecting a site for venipuncture, topically applying to the skin of the subject at the venipuncture site a composition containing a salt of a cation of an element that has an atomic number less than 57 and that, when ionized, has a valence of +1, +2, or +3, thereby causing a vein at the site to enlarge and become more readily accessible for venipuncture, and then puncturing the vein.

12. The method of claim 11 wherein the salt is applied in combination with ethyl or isopropyl alcohol.

13. The method of claim 11 wherein 3 ml or less of the composition is applied to the skin.

14. The method of claim 11 wherein the composition is in the form of a liquid which is impregnated in an absorbent fabric and application of the composition is by contacting the fabric to the skin.

15. The method of claim 11 wherein the cation is magnesium.

16. The method of claim 11 wherein the cation is selected from the group consisting of magnesium, calcium, copper, strontium, and manganese.

17. The method of claim 11 wherein the anion of the salt is selected from the group consisting of sulfate, chloride, citrate, and acetate.

18. The method of claim 11 wherein the composition is applied to the skin by wiping, dabbing, swabbing, or rubbing a fabric swab impregnated with the composition onto the skin.

19. The method of claim 11 wherein the salt compound is applied to the skin in combination with benzalkonium chloride or povidone-iodine.

20. A kit for facilitating venipuncture in an animal or human subject comprising a package housing a container, a composition within the container which composition comprises a salt of a cation of an element that has an atomic number less than 57 and that, when ionized, has a valence of +1, +2, or +3, and an applicator for topically applying the topical composition to the surface of the skin of the subject.

21. The kit of claim 20 which further comprises instructions for topical application of the composition to the skin to prepare a vein for venipuncture.

22. The kit of claim 20 wherein the applicator is a fabric swab that is impregnated with the composition.

23. The kit of claim 22 wherein the container is a sealed foil-lined disposable pouch that is torn open by a user to break the seal and to provide access to the swab.

24. The kit of claim 20 wherein the cation is magnesium.

25. The kit of claim 20 wherein the cation is selected from the group consisting of magnesium, calcium, copper, strontium, and manganese.

26. The kit of claim 20 wherein the anion of the salt is selected from the group consisting of sulfate, chloride, citrate, and acetate.

27. The kit of claim 20 wherein the applicator composition is a fabric swab that, when impregnated with the composition, will deposit the composition on the skin of the subject when wiped, dabbed, swabbed, or rubbed on the skin.
